(19) United States
(12) Patent Application Publication
(10) Pub. No.: US 2002/0037594 A1
(43) Pub. Date: Mar. 28, 2002

(54) Magnetic compound

(76) Inventors: Robert H. Marchessault, Montreal (CA); Dominic H. Ryan, Baie d'Urfe (CA); Virna Veiga, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 09/837,742

(22) Filed: Apr. 18, 2001

Related U.S. Application Data
(60) Provisional application No. 60/198,446, filed on Apr. 19, 2000.

(51) Int. Cl. G01N 33/536

(52) U.S. Cl. 436/536

(57) ABSTRACT

A composition comprising a hydrogel matrix and a particulate magnetic material within the matrix may be employed to entrap different agents, including biological entities such as antibodies and antigens, whereby the composition may be employed in immunoseparation and in diagnostic procedures.

### CROSS REFERENCE TO RELATED APPLICATION

This application claims priority from U.S. Provisional patent application Ser. No. 60/198,446 filed Apr. 19, 2000, and the benefit of 35 USC 119(e).

### BACKGROUND OF THE INVENTION

i) Field of the Invention

This invention relates to magnetic compositions useful in immunoseparation processes and in diagnostic methods, as well as immunoseparation devices and diagnostic kits based on such compositions. (Platsoukas 1987)

ii) Description of Prior Art

Starch is one of the most important energy sources in plants and is considered as an important renewable resource which finds applications in foods, paper, cosmetic and pharmaceuticals products. Common starch consists of a mixture of two main polymers namely amylose and amylopectin. Amylose is the linear fraction of the starch and is composed of about 4000 glucose units joined by - 1,4 links and amylopectin is the branched fraction (−1,6 branch locations) composed of about 500,000 glucose units. The amylose content of hybrid starches is reported to vary from 0-70% (w/w). In most starches, amylose content is 25-30% (w/w). Starch properties are related to the amylose and amylopectin ratio, to their respective molecular weights, and to the cluster arrangement of amylopectin dendrimer branches. Many of the applications of starch depend on its ability to transform into an amorphous gel or paste and to form crystalline complexes with organic molecules.

A “swelling controlled” slow release starch excipient, typically composed of 70% amylose and 30% amylopectin (high amylose starch), has recently been described under the trade name Contramid® (Lenaerts et al. 1991). The manufacturing process involves crosslinking of gelatinized high amylose starch in 4% aqueous NaOH. The resultant purified powder can be described as a starch nanomolecular material which when blended with a pharmaceutical agent and compressed to tablet form provides a near zero-order drug release profile for up to 40 hours (Moussa and Cartlier 1996 and 1997).

### SUMMARY OF THE INVENTION

It is an object of this invention to provide a composition comprising a hydrogel matrix and particulate magnetic material, which composition has various utilities including use in immunoseparation processes, in immunoseparation devices, in diagnostic methods, in diagnostic kits and in xerogels.

In accordance with the invention there is provided a composition comprising a hydrogel matrix and a particulate magnetic material within said matrix.

In another aspect of the invention there is provided an improvement in an immunoseparation process in which an immuno- reactant for the process is entrapped in a composition of the invention.

In yet another aspect of the invention there is provided an improvement in a diagnostic method in which a diagnostic agent is supported in a support material which comprises a composition of the invention.

In still another aspect of the invention there is provided a diagnostic kit comprising a diagnostic agent supported in a composition of the invention.

In still another aspect of the invention there is provided an immunoseparation device comprising an immuno-reactant entrapped in a composition of the invention.

In yet another aspect of the invention there is provided a xerogel of the composition of the invention.

### DESCRIPTION OF PREFERRED EMBODIMENTS

The hydrogel matrix is, more especially, derived from gelatinized starch granules.

Gelatinization of starch granules is an irreversible process of swelling/hydration which melts the crystalline domains and expels the amylose chains while leaving a ghost granule of amylopectin or fragments thereof. The cross-linking effect in the Contramid® process produces a sterically stabilized amylopectin or ghost fragment due to the chemically attached amylose molecules. The purified product when freeze dried or spray-dried is a low crystallinity powder where individual particles will swell in water into a particulate hydrogel.

This particulate hydrogel matrix provides a high surface area of carbohydrate host material which is suited to the in situ synthesis of submicron particles. In accordance with the invention, a particulate magnetic material is incorporated into the matrix of particulate starch gel to yield a magnetic carbohydrate mass. This swollen mass, preferentially of superparamagnetic character, presents a permeable and accessible inner surface. Such a surface is a “cage” for antigens or antibodies, chemically or physically attached, in a designed immuno-separation process (Rembaum 1982). The same high surface area hydrogel particles are ideal for diagnostic test kits for diseases and physiological states such as AIDS, pregnancy, etc (Karlsson and Platt 1991).

Thus the granules may comprise a framework of amylopectin from which amylose chains have been expelled; the matrix being permeable and having accessible interior surfaces defining a cage for physical or chemical entrapment of an immuno-reactant or a diagnostic agent, for example antibodies or antigens.

The composition of the invention may be produced by in situ formation of the particulate magnetic material in the matrix. The particulate magnetic material may, in particular, comprise iron particles having a particle size of 1 to 100 nm.

In a preferred embodiment the matrix is a Contramid® matrix. Iron is introduced inside the Contramid® matrix by immersion in a ferrous chloride solution. Subsequently the iron is precipitated and finally oxidized. The resulting superparamagnetic material was characterized using three techniques: X-ray diffraction, vibrating sample magnetometry and Mossbauer spectroscopy.

In the freeze-dried or spray-dried form the powder is a xerogel and this represents a further embodiment of the invention.

The particulate magnetic material is suitably present in an amount of 1 to 50%, by weight, preferably 10 to 20%, by weight based on the total weight of the xerogel containing the particulate magnetic material.

### MATERIALS AND METHODS

### Materials

Contramid® powder, kindly supplied by Rougier Inc., was prepared using a patented process (Mateescu et al., 1991) based on epichlorohydrin cross-linking followed by purification and spray-drying from water to form a coarse powder. A solution of solid iron (II) chloride tetra hydrate (Aldrich Chemical Company, Inc.) was employed as iron source. Pellets of sodium hydroxide and hydrogen peroxide 30% solution (both from ACP Chemicals Inc.) were also used during the synthesis process.

### In Situ Synthesis of the Magnetic Material

A suspension of 0.75 g of Contramid® in 25 mL of water (2.9% wt. starch suspension) was slowly added to 250 mL of a degassed and constantly stirred 0.5 M solution of FeCl₂-4H₂O. The system was stirred, while keeping the N₂ bubbling, for another 2 hours. After this period the starch gel with ferrous ions embedded was separated by centrifugation and dispersed in 250 mL of distilled water. Afterwards, 200 mL of a 0.5 M NH₄OH solution were added to the yellowish-brown starch slurry, which immediately became greenish-brown. Finally, the mixture was placed into a 65±5° C. water bath and 10 mL of hydrogen peroxide (10% wt.) were added dropwise. The color became reddish-brown and, once the last drop added, the solution was removed from the heat source and stirred for 30 min. to complete the oxidation process. The final product was neutralized with acid to pH7 and separated from the suspension by centrifugation and washing. Freeze-drying was used to dry the washed product.

If a higher iron content is desired, the entire operation is repeated with the above product prior to freeze-drying. In this case five cycles were performed and the collected freeze-dried samples were characterized.

### Determination of the Iron Content

Guelph Chemical Laboratories provided the micro-analytical determination of the iron content for the samples.

### X-Ray Diffraction (XRD)

A Rigaku powder diffractometer with a Cu rotating anode generator and a graphite monochromator was used to analyze the products. The curves of relative intensities as a function of the Bragg angle, as well as d-spacing are provided by the printout.

### Vibrating Sample Magnetometry (VSM)

Approximately 20 mg of the different products at room temperature were vibrated in a magnetic field varying from −1.5 T to 1.5 T. The data of magnetization as a function of the applied field were plotted and employed for further calculations.

### Mössbauer Spectroscopy (MS)

Mössbauer spectra at room temperature were obtained with a conventional constant-acceleration spectrometer in transmission geometry and with a 1 GBq 57CoRh source. The spectra for the first, third and fifth cycles were obtained and fitted using a standard Mössbauer fitting program.

### BRIEF DESCRIPTION OF DRAWINGS

FIG. 1. Complete magnetization curves at room temperature for the samples after the first (±), third (A) and fifth (a) oxidation cycle.

FIG. 2. Initial magnetization curves at room temperature for the samples after the first (±), third (A) and fifth (a) oxidation cycle.

FIG. 3. Particle size distributions calculated from the VSM data for the first (±), third (A) and fifth (−) oxidation cycle.

FIG. 4. Mossbauer spectra at room temperature for the samples after the fifth (a), third (b) and first (c) oxidation cycle.

### RESULTS

According to the reported decrease of the iron content (42.56%, 39.08% and 29.92% for the first, third and fifth cycle respectively) we can state that the iron compounds are not chemically bound to the polymer matrix. It seems that physical interactions between both components of the composite material are not strong enough to prevent the mechanical loss of magnetic phase when multiple oxidation cycles are performed.

Furthermore, the X-ray diffraction patterns were very difficult to analyze since multiple iron compounds seem to be present and some peaks could correspond to more than one of them. Only the signals at 2θ=35.09° (d=2.56 Å) and 2θ=62.820 (d=1.48 Å) are constantly present in the diffractograms of the different cycles. They can be associated not only with magnetite (Fe₃O₄) and maghemite (γ−Fe₂O₃), very important in the final magnetic properties due to their high specific magnetizations, but also with other forms of iron oxides, such as haematite (α-Fe₂O₃). Oxy-hydroxy products such as feroxyhite (δ-FeOOH) and lepidocrocite (γ-FeOOH), are likely in the case of the first cycle. Additional peaks characteristic of the completely oxidized material appear only as a result of more than one oxidation step (Powers 1975).

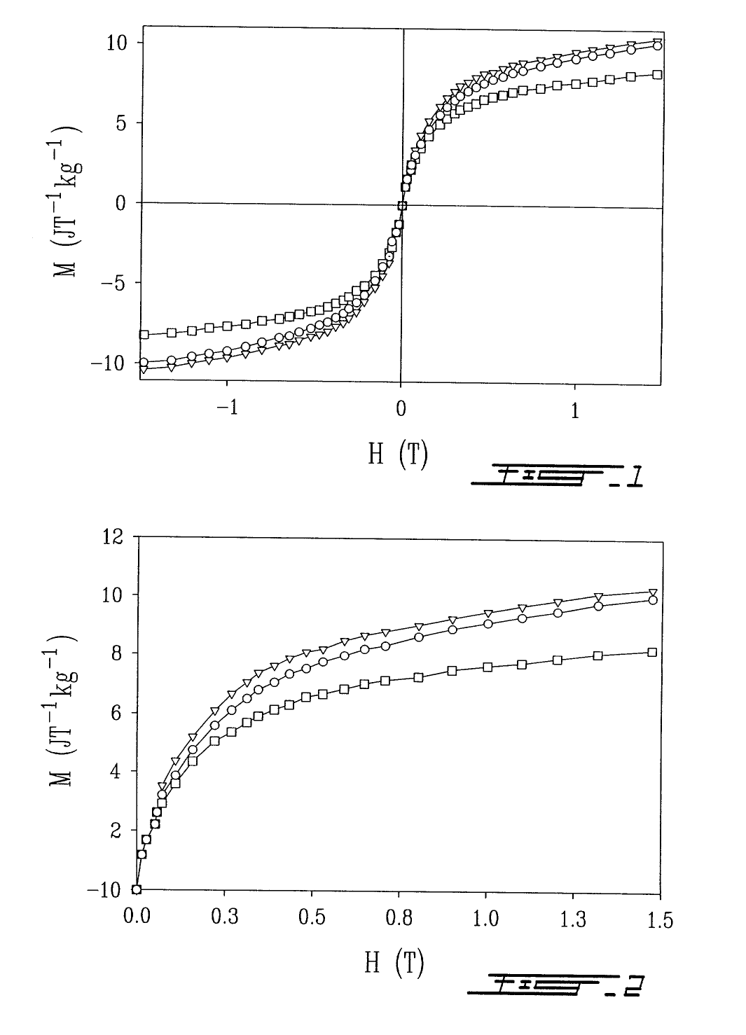

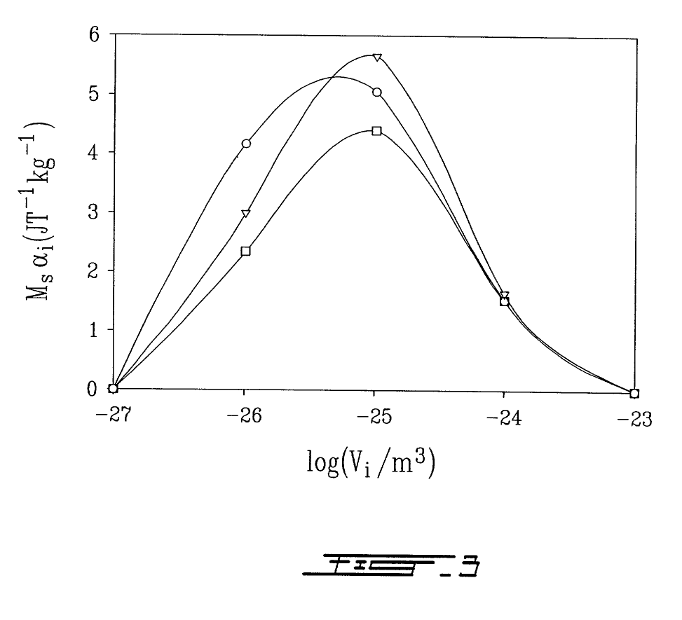

All samples showed good magnetic response under the influence of a permanent magnet; and the magnetization curves shown in FIG. 1 and 2 proved their superparamagnetism. High magnetizations, above 6 JT-1 kg-1, are present for relatively small magnetic field values, below 0.5 T, while neither coercitivity nor remanence were observed. The saturation magnetization increases slightly from the first to the third cycle, despite the decreasing iron content. This can be explained by the chemical transformation occurring during the oxidation process, which leads to the conversion of some weakly ferrimagnetic or antiferromagnetic compounds (e.g. feroxyhite or lepidocrocite) into strongly ferrimagnetic compounds (e.g. magnetite or maghemite). However, a decrease of the saturation magnetization is observed from the third to the fifth cycle, in agreement with the decrease of the iron content, suggesting that no more than three cycles should be performed in order to obtain a product with maximum magnetic properties.

These results allow us to assume a simple model to estimate the size of the particles from the VSM data plotted in FIG. 2. The very small particles (smaller than the critical size) are considered as non-interacting ferrimagnetic domains with a certain size distribution. Their behavior resembles that of the classic paramagnetic gas and can be described by the Langevin function L (Chikazumi, 1997):

$$M = {M}_{s} {\sum }_{i} {α}_{i} L ( \frac{{m}_{s} {V}_{i} H}{kT} )$$

where M is the magnetization obtained for the applied magnetic field H; Ms is the saturation magnetization; and (xi is the fraction of particles with volume Vi and specific spontaneous magnetization ms at the temperature T.

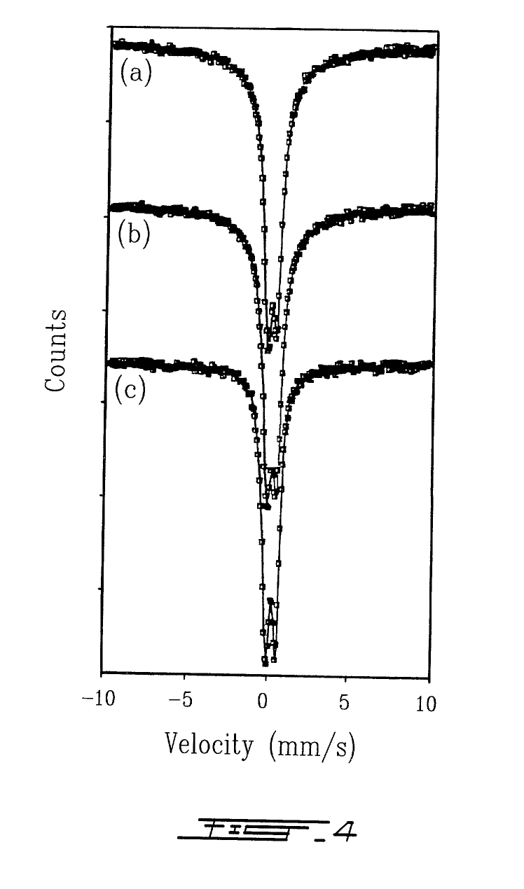

FIG. 3 shows the size distribution curves obtained when assuming ms =5·105 JT-1 m-3 (value for magnetite). The three cycles have similar distributions, principally for larger particle volumes. The chemical transformations occurring during the consecutive oxidation cycles cause changes in the shapes of the particles, rather than changes in their sizes (Ugelstad et al. 1985).

The volumes of the particles contributing to the VSM curves are lower than 10-23 m3, which means that their diameters are lower than 27 nm if they are assumed as spherical. This value is slightly higher than the sizes observed by TEM. The initial parts of the magnetization curves of FIG. 2 are associated with the orientation of the largest particles at low applied fields and that is why the magnetization increases sharply. However, the model considered could lead to exaggerated estimates of the maximum volume since we are disregarding particle interactions and the orientation effect of the local particle fields, which are particularly important for small values of applied field and for particles with large volumes.

The products of the different cycles appear superparamagnetic on the time scale of the VSM measurements, i.e. about one second. Furthermore, particles are so small that the relaxation time for the change in magnetization direction is less than the lifetime of the nuclear excited state (˜10-7 s). As a consequence, the magnetic hyperfine splittings characteristic of the Mossbauer effect for the iron compounds are not observed, and the spectra collapse to doublet resonant peaks.

FIG. 4 illustrates the Mossbauer spectra for the three cycles studied. After the first oxidation process a doublet with a quadrupole splitting of ˜0.68 mm/s is observed. For the third and fifth cycles the quadrupole splittings were ˜0.73 mm/s and ˜0.66 mm/s respectively. The differences are a result of the multiple iron compounds present in the samples.

### CONCLUSION

The proposed in situ synthesis of ferrites is an effective method to produce cross-linked high amylose starch with superparamagnetic properties, although it has been very difficult to control the oxidation process in order to obtain more selectively the optimal magnetic phases (magnetite and maghemite) in adequate proportions. Nevertheless, the magnetic behavior of the final products is appropriate for applications related to separation of bioactive molecules using molecular recognition methods.

The method employed can be modified with the addition of fresh ferrous solutions on each oxidation cycle. Preliminary studies have demonstrated that this modification avoids the undesirable decrease of the iron content, thereby contributing to an improvement in the overall magnetic response for each cycle.

Thus in summary cross-linked high amylose starch with magnetic properties were synthesized via in situ formation of iron oxides inside the polymer matrix. Precipitation and multiple oxidation of ferrous ions were performed. The iron content analysis revealed decay from one oxidation cycle to the next one. X-ray diffractograms, magnetization curves and Mossbauer spectra were also recorded for the characterization of the magnetic phase. The products exhibit superparamagnetic properties due to the presence of ferrimagnetic nanoparticles, although some other iron compounds are also present.

We claim:

1. A composition comprising a hydrogel matrix and a particulate magnetic material within said matrix.

2. A composition according to claim 1 wherein said matrix is derived from gelatinized starch granules.

3. A composition according to claim 2 in which said granules comprise a framework of amylopectin from which amylose chains have been expelled.

4. A composition according to claim 3 wherein said particulate material is super paramagnetic.

5. A composition according to claim 4, wherein said matrix is permeable and has accessible interior surfaces defining a cage for physical or chemical entrapment of antigens or antibodies.

6. In an immunoseparation process employing an immuno-reactant, the improvement wherein the immuno-reactant is entrapped in a composition as defined in claim 1.

7. In a diagnostic method in which a diagnostic agent is supported in a support material, the improvement wherein the support material is a composition as defined in claim 1.

8. A diagnostic kit comprising a diagnostic agent supported in a composition as defined in claim 1.

9. An immunoseparation device comprising an immuno-reactant entrapped in a composition as defined in claim 1.

10. A xerogel of the composition of claim 1.
